(19) United States
(12) Patent Application Publication
(10) Pub. No.: US 2008/0228250 A1
(43) Pub. Date: Sep. 18, 2008

(54) PADDLE LEAD COMPRISING OPPOSING DIAGONAL ARRANGEMENTS OF ELECTRODES AND METHOD FOR USING THE SAME

(76) Inventor: Y. Eugene Mironer, Spartanburg, SC (US)

Correspondence Address: ADVANCED NEUROMODULATION SYSTEMS, INC., 6901 PRESTON ROAD, PLANO, TX, 75024, US

(21) Appl. No.: 12/047,542

(22) Filed: Mar. 13, 2008

Related U.S. Application Data
(60) Provisional application No. 60/895,175, filed on Mar. 16, 2007.

(51) Int. Cl. A61N 1/00

(52) U.S. Cl. 607/117

(57) ABSTRACT

In one embodiment, a paddle lead for electrical stimulation of a patient comprises a lead body of insulative material; a plurality of electrical terminals disposed at a proximal end of the lead body; a paddle structure disposed at distal end of the lead body; a plurality of electrodes disposed on the paddle structure; a plurality of conductors disposed within the lead body, wherein the plurality of conductors electrically couple the plurality of terminals with the plurality of electrodes; wherein the plurality of electrodes are arranged in at least two sets that are disposed in opposing generally diagonal arrangements relative to a longitudinal axis of the paddle structure.

### CROSS-REFERENCE TO RELATED APPLICATIONS

This application claims the benefit of U.S. Provisional Application No. 60/895,175, filed Mar. 16, 2007, the disclosure of which is incorporated herein by reference.

### BACKGROUND

The present application is directed to a paddle lead for electrical stimulation of a patient in which electrodes on the paddle structure of the lead are preferably configured in two opposing diagonal sets.

Application of electrical fields to spinal nerve roots, spinal cord, and other nerve bundles for the purpose of chronic pain control has been actively practiced for some time. While a precise understanding of the interaction between the applied electrical energy and the nervous tissue is not fully appreciated, it is known that application of an electrical field to spinal nervous tissue (i.e., spinal nerve roots and spinal cord bundles) can effectively mask certain types of pain transmitted from regions of the body associated with the stimulated nerve tissue. Specifically, applying electrical energy to the spinal cord associated with regions of the body afflicted with chronic pain can induce “paresthesia” (a subjective sensation of numbness or tingling) in the afflicted bodily regions. Thereby, paresthesia can effectively mask the transmission of non-acute pain sensations to the brain.

A neurostimulation system typically includes a pulse generator and one or several leads. The pulse generator is the device that generates the electrical pulses. The pulse generator is typically implanted within a subcutaneous pocket created under the skin by a physician. The leads are used to conduct the electrical pulses from the implant site of the pulse generator to the targeted nerve tissue. The leads typically include a lead body of an insulative polymer material with embedded wire conductors extending through the lead body. Electrodes on a distal end of the lead body are coupled to the conductors to deliver the electrical pulses to the appropriate nerve tissue.

Percutaneous leads and laminotomy leads are the two most common types of lead designs that provide conductors that deliver stimulation pulses from an implantable pulse generator (IPG) to distal electrodes adjacent to the nerve tissue. As shown in FIG. 1A, conventional percutaneous lead 100 includes electrodes 101 that substantially conform to the body of the body portion of the lead. Due to the relatively small profile of percutaneous leads, percutaneous leads are typically positioned above the dura layer through the use of a Touhy-like needle. Specifically, the Touhy-like needle is passed through the skin, between desired vertebrae to open above the dura layer for the insertion of the percutaneous lead.

The specific implantation location of electrodes of a percutaneous or other lead affects the patient's experience of paresthesia. Each exterior region, or each dermatome, of the human body is associated with a particular longitudinal spinal position. The head and neck regions are associated with levels C2-C8, the back regions extends from levels C2-S3, the central diaphragm is associated with spinal nerve roots between levels C3 and C5, the upper extremities are correspond to levels C5 and T1, the thoracic wall extends from levels T1 to T11, the peripheral diaphragm is between levels T6 and T11, the abdominal wall is associated with levels T6-L1, lower extremities are located from levels L2 to S2, and the perineum from levels L4 to S4. By example, to address chronic pain sensations that commonly focus on the lower back and lower extremities, a specific energy field can usually be applied to a region between levels T8 and T10.

Additionally, positioning of an applied electrical field relative to a physiological midline is also important. For example, “bilateral pain” is used to refer to pain which affects both sides of the patient's body. For example, lower back pain is often considered bilateral in nature. Bilateral pain is most effectively addressed by the use of a cathode and anode combination positioned immediately above the physiological midline of the patient.

However, placement of electrodes of a percutaneous lead directly over the physiological midline of the patient is not necessarily easily accomplished. For example, the physiological midline can vary from the anatomical midline and its exact location is not known before implantation of the lead. Also, the ability to laterally adjust the position of the lead within the epidural space is constrained due to the nature of percutaneous implantation procedure. Accordingly, in one conventional practice, two percutaneous leads are implanted on either side of the physiological midline. The two percutaneous leads allow electrical pulses to be applied to neural tissue on either side of the physiological midline and, typically, enable the patient to experience paresthesia at the desired location. However, the use of two percutaneous leads is not ideal. The use of two percutaneous leads is more expensive, involves greater complexity in the implantation procedure, requires additional power for the application of the electrical pulses, and does not necessarily provide optimal paresthesia coverage in the patient as compared to the use of a single percutaneous lead placed directly over the physiological midline.

FIG. 1B depicts conventional laminotomy or paddle lead 150 which has a paddle configuration and typically possesses a plurality of electrodes arranged in one or more columns. One common type of paddle lead disposes three columns of electrodes on the paddle structure in a “tripole” manner. Tripole stimulation generally refers to stimulation applied using a set of adjacent electrodes where first and second active electrodes are positioned on opposite sides of a third active electrode. The third electrode is controlled to function as a cathode while the first and second electrodes are controlled to function as anodes (at independent amplitudes). Such tripole stimulation has been reported to facilitate “steering” of the stimulation along an axis between the first, second, and third electrodes. The steering is believed to be capable of compensating for implantation of the middle column of electrodes off center relative to the physiological midline. However, tripole stimulation has been reported to require significantly more power than conventional bipolar stimulation (i.e., a single cathode and anode combination). Additionally, if a tri-pole paddle becomes appreciably translated left or right within the epidural space, the effectiveness of the stimulation therapy has been observed to be significantly reduced.

### SUMMARY

In one embodiment, a paddle lead for electrical stimulation of a patient comprises a lead body of insulative material; a plurality of electrical terminals disposed at a proximal end of the lead body; a paddle structure disposed at distal end of the lead body; a plurality of electrodes disposed on the paddle structure; a plurality of conductors disposed within the lead body, wherein the plurality of conductors electrically couple the plurality of terminals with the plurality of electrodes; wherein the plurality of electrodes are arranged in at least two sets that are disposed in opposing generally diagonal arrangements relative to a longitudinal axis of the paddle structure.

The foregoing has outlined rather broadly certain features and/or technical advantages in order that the detailed description that follows may be better understood. Additional features and/or advantages will be described hereinafter which form the subject of the claims. It should be appreciated by those skilled in the art that the conception and specific embodiment disclosed may be readily utilized as a basis for modifying or designing other structures for carrying out the same purposes. It should also be realized by those skilled in the art that such equivalent constructions do not depart from the spirit and scope of the appended claims. The novel features, both as to organization and method of operation, together with further objects and advantages will be better understood from the following description when considered in connection with the accompanying figures. It is to be expressly understood, however, that each of the figures is provided for the purpose of illustration and description only and is not intended as a definition of the limits of the appended claims.

### BRIEF DESCRIPTION OF THE DRAWINGS

FIGS. 1A and 1B respectively depict conventional percutaneous and paddle leads.

FIGS. 2-7 depict respective paddle leads according to some representative embodiments.

FIG. 8 depicts a stimulation system according to one representative embodiment.

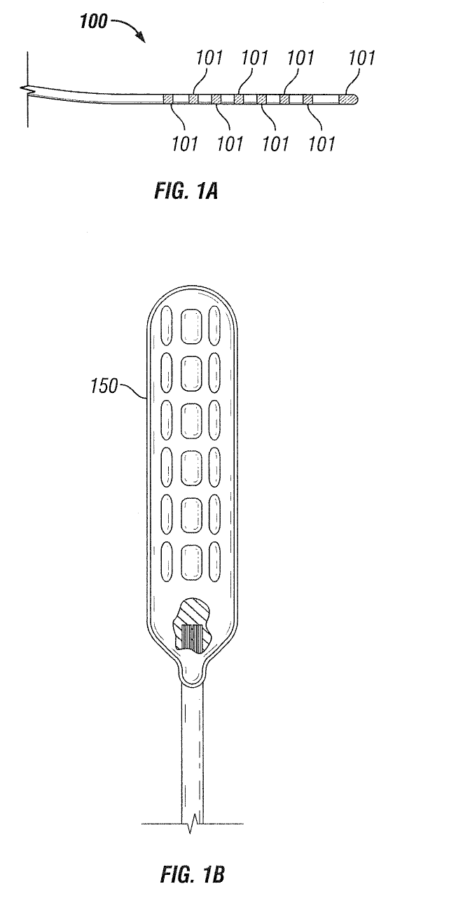

### DETAILED DESCRIPTION

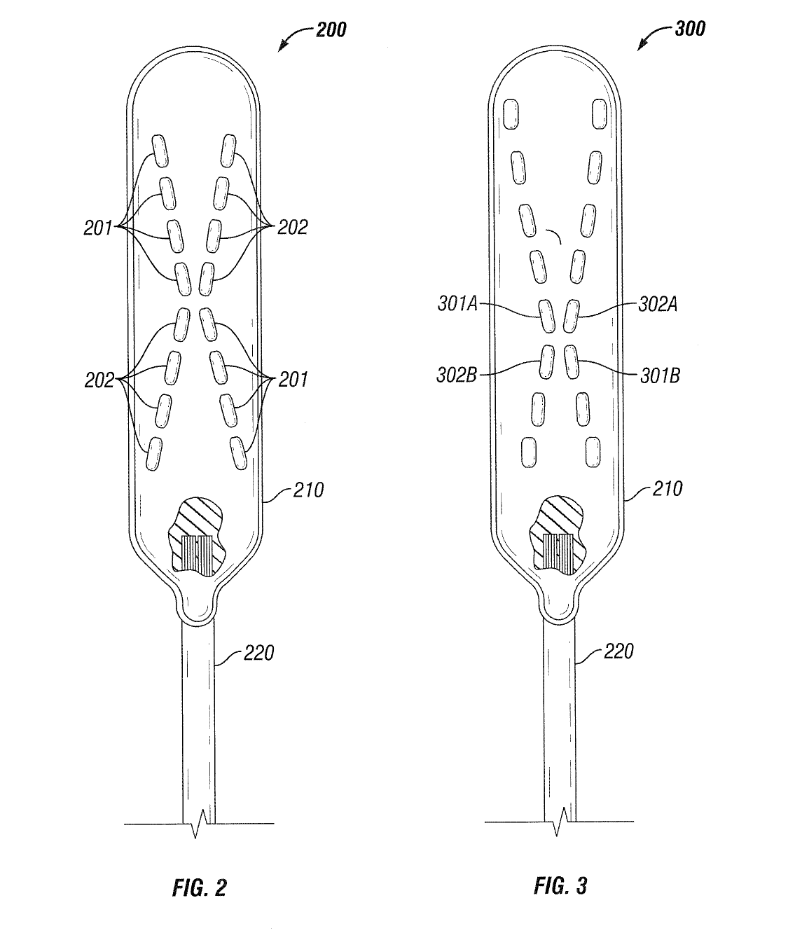

FIG. 2 depicts paddle 200 according to one representative embodiment. Lead 200 comprises lead body 220 of a suitable insulative material such as polyurethanes, silicone-based materials (e.g., PurSil™ and CarboSil™), polyethylene, polyimide, polyvinylchloride, PTFT, EFTE, etc.

Wire conductors (not shown) are preferably encapsulated or embedded within lead body 220. Each conductor is formed of a conductive material that exhibits desired mechanical properties of low resistance, corrosion resistance, flexibility, and strength. While conventional stranded bundles of stainless steel, MP35N, platinum, platinum-iridium alloy, drawn-brazed silver (DBS) or the like can be used, a preferred embodiment of the present invention uses conductors formed of multi-strands of drawn-filled tubes (DFT). Each strand is formed of a low resistance material and is encased in a high strength material (preferably, metal). A selected number of “sub-strands” are wound together and then coated with an insulative material. With regard to the operating environment of the present invention, such insulative material protects the individual conductors if its respective sheath was breached during use.

Any suitable process for fabricating lead body 220 may be utilized. For example, the fabrication process may comprise (i) extrusion of insulative material, (ii) followed by wrapping of insulative coated conductive wires, (iii) extrusion of additional insulative material, and (iii) application of heat and pressure to fuse the various insulative material(s) using shrink wrap material. Such fabrication processes are discussed in greater detail in U.S. patent application Ser. No. 10/630,233, filed Jul. 29, 2003, entitled “SYSTEM AND METHOD FOR PROVIDING A DUAL WRAP LEAD BODY WITH INNER AND OUTER EXTRUSION,” which is incorporated herein by reference.

Laminotomy lead 200 comprises paddle structure 210 adapted for implantation within a patient proximate to neural tissue to be stimulated. Paddle structure 210 is preferably fabricated using a biostable, biocompatible insulative material such as polyurethanes, silicone-based materials, and the like. The material selected for paddle structure 210 preferably provides a flexible and durable (i.e., fatigue resistant) exterior structure for the components of lead 200. Additionally, the material electrically insulates electrodes 201 from each other. A nylon mesh, a fiberglass substrate, or the like (not shown) can be internalized within the paddle structure 1100 to increase its overall rigidity and/or to cause paddle structure 1100 to assume a prescribed cross-sectional form.

As shown in the embodiment of FIG. 2, paddle structure 210 comprises a plurality of electrodes 201 and 202. Each electrode 201 and 202 is electrically coupled through a wire conductor within lead body to a respective terminal (not shown) on the proximal end of lead body 220. In the embodiment of FIG. 2, sixteen total electrodes 201 and 202 are disposed on paddle structure 200, although any suitable number of electrodes could be provided. In some representative embodiments, the spacing between adjacent electrodes 201 and adjacent electrodes 202 is preferably between approximately 1.0 mm to 1.5 mm, although greater or lesser distances could be employed.

The electrodes of paddle structure 210 are arranged in two diagonal sets of electrodes 201 and 202. Additionally, the sets of electrodes 201 and 202 are oriented in opposing directions. The first set of electrodes 201 are disposed on paddle structure 210 beginning at the left side of the distal end of paddle structure 210 and ending at the right side of the proximal end of paddle structure 210. The second set of electrodes 202 are disposed on paddle structure beginning at the right side of the distal end of paddle structure 210 and ending at the left side of the proximal end of paddle structure 210. In the embodiment of FIG. 2, the two sets of electrodes 201 and 202 intersect at the middle of paddle structure 200.

The arrangement of electrodes on paddle structure 210 is advantageous for stimulating neural tissue near the physiological midline of the patient (e.g., to treat bilateral lower back pain in an effective manner). Specifically, no matter the orientation and lateral position of paddle structure 210 within the epidural space, at least one pair of electrodes 201 or 202 will be positioned (i) immediately to the left and right of and (ii) immediately above and below the physiological midline. Accordingly, the locus of stimulation will include neural tissue at the physiological midline. Due to the ability to obtain suitably positioned electrodes relative to the midline, if paddle structure 210 migrates after implantation, revision of paddle lead 200 (repositioning or removal and replacement) is not necessary. Instead, another pair of electrodes on paddle structure 200 can be selected for use by the IPG to deliver the electrical pulses to neural tissue at the physiological midline.

In contrast, conventional paddle structures having three linear electrode columns can be offset with respect to the physiological midline. That is, the electrodes of the middle column can be positioned laterally away from the physiological midline. To compensate for such an offset, tripole stimulation can be applied on a conventional paddle structure. However, such tripole stimulation is less energy efficient and does not necessarily produce the same coverage as produced by a bipolar pair of electrodes positioned over the physiological midline. Moreover, if the middle column is sufficiently offset from the physiological midline, tripole stimulation may not be able to achieve effective bilateral paresthesia in the patient.

Similarly, the ability to obtain suitably positioned electrodes enables the implantation procedure to be simplified. In conventional implantation paddle procedures, a partial laminectomy is performed where certain vertebral tissue is removed to allow both access to the dura and proper positioning of a laminotomy lead. Due to the invasiveness of such conventional procedures, the patient is given anesthesia. After the lead is placed within the epidural space but before the surgical procedure is completed, the anesthesia is typically reduced to allow the patient to regain consciousness. At that time, trial stimulation is provided via the implanted paddle and the patient is able to indicate whether the stimulation produces paresthesia at the appropriate bodily regions. Specifically, the patient feedback is necessary in conventional procedures to ensure that the paddle is properly positioned and, hence, can be used to provide an effective treatment for the patient. When paddles according to representative embodiments are employed, the surgeon need not cause the patient to regain consciousness during the surgical procedure. Specifically, because the diagonal sets of electrodes ensure that an electrode pair will be properly positioned relative to the physiological midline, the patient feedback is not needed.

Additionally, the use of electrodes disposed in a diagonal arrangement according to some representative embodiments is advantageous for selectively activating small diameter nerve fibers associated with the neuropathic pain experienced by the patient. Furthermore, the presence of two diagonal sets of electrodes 201 and 202 enables paresthesia to be experienced in a more equalized manner. That is, the clinician may select between anode and cathode combinations in both diagonal sets to identify the anode and cathode combination that provides equalized paresthesia on both sides of the patient's body.

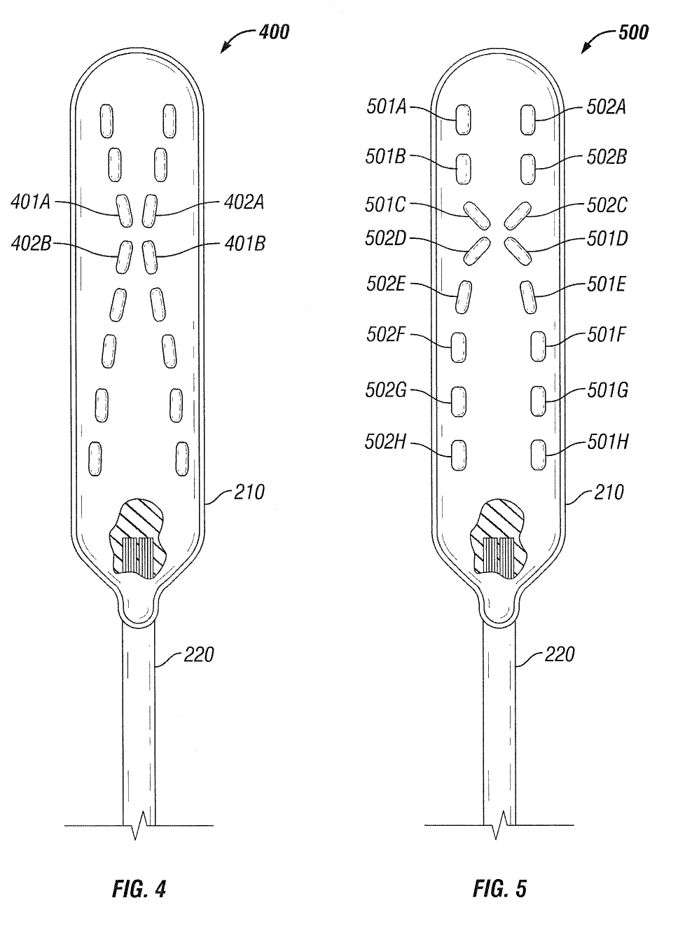

FIG. 3 depicts paddle lead 300 according to another representative embodiment. Paddle lead 300 comprises two sets of generally diagonally oriented sets of electrodes. The two sets of electrodes are disposed in opposing directions relative to each other. Paddle lead 300 differs from paddle lead 200 in several respects. In paddle lead 300, the intersection (denoted by electrodes 301a, 302a, 301b, and 302b) of the two sets of electrodes occurs closer to the proximal end of paddle structure 210. Additionally, the two sets of electrodes do not possess a strictly linear arrangement. The sets of electrodes are generally diagonal, but possess a slight curve between the distal and proximal ends of paddle structure 210. Paddle lead 400 as shown in FIG. 4 is similar to paddle lead 300 except the two sets of electrodes of paddle lead 400 are positionally inverted relative to the two sets of electrodes of paddle lead 300. That is, the intersection (denoted by electrodes 401a, 402a, 402a, and 402b) of the two sets of electrodes of paddle lead 400 occurs closer to the distal end of paddle structure 210.

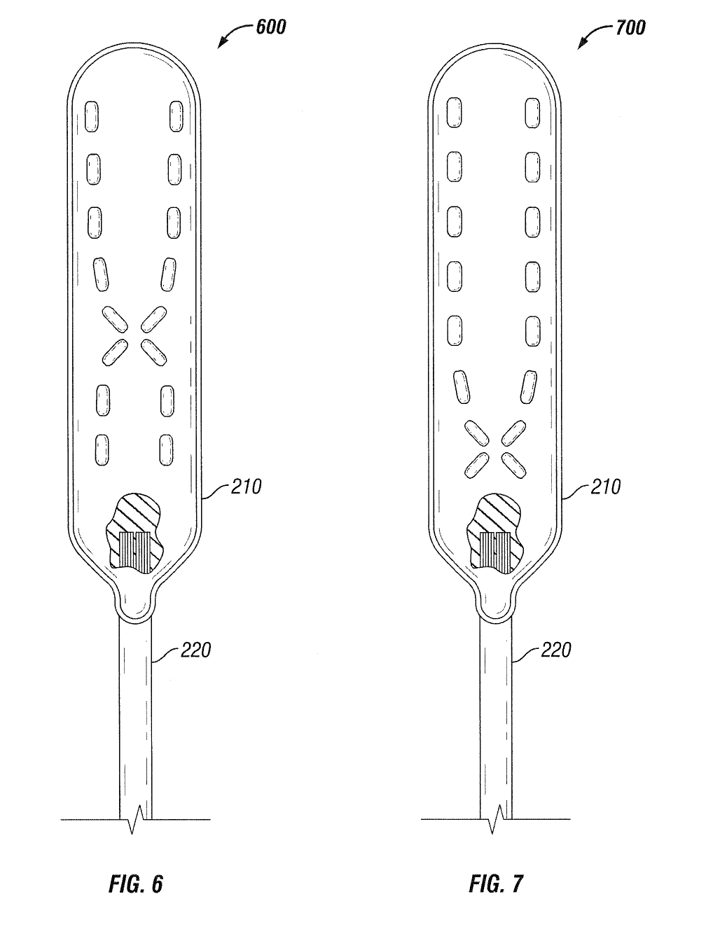

FIGS. 5 and 6 respectively depict paddle leads 500 and 600 according to some representative embodiments. As shown in FIG. 5, the two sets of electrodes generally possess a diagonal arrangement. Also, the two sets of electrodes 501a-501h and 502a-502h of paddle lead 500 possess greater differences in the orientation of the respective electrodes. As shown in FIG. 5, electrodes 501c, 501d, 502c, and 502d possess a greatest angle relative to the longitudinal axis. Electrodes 501e and 502e possess a smaller angle. Electrodes 501a, 502a, 501b, 502b, 501f, 502f, 501g, 502g, 501h, and 502h are parallel or substantially parallel to the longitudinal axis. The arrangement electrodes of paddle lead 600 is substantially similar to the arrangement of electrodes of paddle lead 500 except that the electrodes of paddle lead 600 are positionally inverted relative to the electrodes of paddle lead 500.

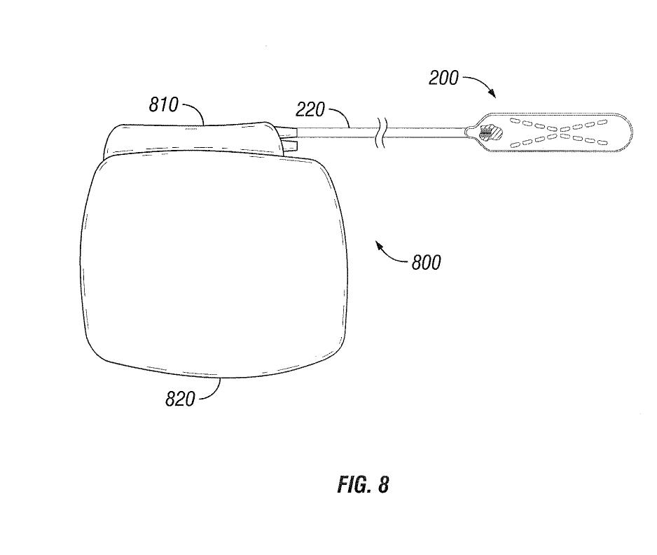

FIG. 7 depicts paddle lead 700 according to another representative embodiment. Paddle lead 700 is adapted for introduction in a retrograde manner for stimulation of neural tissue associated with pelvic regions of a patient. The electrodes of paddle lead 700 are generally diagonal. The intersection of the two sets of electrodes of paddle lead 700 occurs at the very proximal end of paddle structure 210.

FIG. 8 depicts paddle lead 200 coupled to implantable pulse generator (IPG) 800 according to one representative embodiment. Paddle leads 300-700 could be employed in lieu of paddle lead 200 according to other embodiments. An example of a commercially available IPG that could be employed according to one embodiment is the Eon® Rechargeable IPG available from Advanced Neuromodulation Systems, Inc. As shown in FIG. 8, paddle lead 200 is coupled to header 810 of generator 800. Header 810 electrically couples to one or more respective leads 220 (or an extension lead for coupling to lead 220 through an extension connector). Also, header 810 electrically couples to internal components contained within the sealed portion 820 of IPG 800. The sealed portion 820 contains the pulse generating circuitry, communication circuitry, control circuitry, and battery (not shown) within an enclosure to protect the components after implantation within a patient. The control circuitry controls the pulse generating circuitry to apply varying pulses to the patient via electrodes 201 and/or 202 of paddle lead 200 according to multiple parameters (e.g., amplitude, pulse width, frequency, etc.). The parameters are set by an external programming device (not shown) via wireless communication with IPG 800.

Although certain representative embodiments and advantages have been described in detail, it should be understood that various changes, substitutions and alterations can be made herein without departing from the spirit and scope of the appended claims. Moreover, the scope of the present application is not intended to be limited to the particular embodiments of the process, machine, manufacture, composition of matter, means, methods and steps described in the specification. As one of ordinary skill in the art will readily appreciate when reading the present application, other processes, machines, manufacture, compositions of matter, means, methods, or steps, presently existing or later to be developed that perform substantially the same function or achieve substantially the same result as the described embodiments may be utilized. Accordingly, the appended claims are intended to include within their scope such processes, machines, manufacture, compositions of matter, means, methods, or steps.

1. A paddle lead for electrical stimulation of a patient, the lead comprising:
a lead body of insulative material;
a plurality of electrical terminals disposed at a proximal end of the lead body;
a paddle structure disposed at distal end of the lead body;
a plurality of electrodes disposed on the paddle structure;
a plurality of conductors disposed within the lead body, wherein the plurality of conductors electrically couple the plurality of terminals with the plurality of electrodes;
wherein the plurality of electrodes are arranged in at least two sets that are disposed in opposing generally diagonal arrangements relative to a longitudinal axis of the paddle structure.

2. The paddle lead of claim 1 wherein the two sets intersect at a location at the approximate center of the paddle structure.

3. The paddle lead of claim 1 wherein the two sets intersect at a location at a proximal portion of the paddle structure.

4. The paddle lead of claim 1 wherein the two sets intersect at a location at a distal portion of the paddle structure.

5. The paddle lead of claim 1 wherein one or more electrodes, adjacent to a location where the two sets intersect, are disposed at an angle relative to the longitudinal axis that is greater than an angle of other electrodes of the two sets relative to the longitudinal axis.

6. The paddle lead of claim 1 wherein the respective electrodes of the two sets form at least one slight curve between a distal and proximal end of the paddle structure.

7. A method of providing a neurostimulation therapy to a patient, comprising;
implanting a paddle lead within the epidural space of the patient, wherein the paddle lead comprises: a lead body of insulative material; a plurality of electrical terminals disposed at a proximal end of the lead body; a paddle structure disposed at distal end of the lead body; a plurality of electrodes disposed on the paddle structure; a plurality of conductors disposed within the lead body, wherein the plurality of conductors electrically couple the plurality of terminals with the plurality of electrodes; wherein the plurality of electrodes are arranged in at least two sets that are disposed in opposing generally diagonal arrangements relative to a longitudinal axis of the paddle structure;
identifying an anode and cathode combination within the plurality of electrodes located immediately proximate to a physiological midline of neural tissue;
providing bipolar stimulation to the patient using the identified anode and cathode combination.

8. The method of claim 7 wherein the providing bipolar stimulation provides substantially equalized paresthesia coverage on both sides of the patient's body.

9. The method of claim 8 further comprising:
selecting the anode and cathode combination from the at least two sets of electrodes by determining which set provides more equalized paresthesia coverage.

10. The method of claim 7 wherein the providing bipolar stimulation treats lower back pain of the patient.

11. The method of claim 7 wherein the two sets intersect at a location at the approximate center of the paddle structure.

12. The method of claim 7 wherein the two sets intersect at a location at a proximal portion of the paddle structure.

13. The method of claim 7 wherein the two sets intersect at a location at a distal portion of the paddle structure.

14. The method of claim 7 wherein one or more electrodes, adjacent to a location where the two sets intersect, are disposed at an angle relative to the longitudinal axis that is greater than an angle of other electrodes of the two sets relative to the longitudinal axis.

15. The method of claim 7 wherein the respective electrodes of the two sets form at least one slight curve between a distal and proximal end of the paddle structure.

16. A system for electrical stimulation of a patient, the system comprising:
an implantable pulse generator for generating electrical pulses; and
a paddle lead for delivering electrical pulses to neural tissue of the patient, the paddle comprising:
a lead body of insulative material;
a plurality of electrical terminals disposed at a proximal end of the lead body;
a paddle structure disposed at distal end of the lead body;
a plurality of electrodes disposed on the paddle structure; and
a plurality of conductors disposed within the lead body, wherein the plurality of conductors electrically couple the plurality of terminals with the plurality of electrodes, wherein the plurality of electrodes are arranged in at least two sets that are disposed in opposing generally diagonal arrangements relative to a longitudinal axis of the paddle structure.

17. The system of claim 16 wherein the two sets of electrodes intersect at a location at the approximate center of the paddle structure.

18. The system of claim 16 wherein the two sets intersect at a location at a distal portion of the paddle structure.

19. The system of claim 16 wherein the respective electrodes of the two sets form at least one slight curve between a distal and proximal end of the paddle structure.

20. The system of claim 16 wherein one or more electrodes, adjacent to a location where the two sets intersect, are disposed at an angle relative to the longitudinal axis that is greater than an angle of other electrodes of the two sets relative to the longitudinal axis.
